(19) United States
(12) Patent Application Publication
(10) Pub. No.: US 2015/0040908 A1
(43) Pub. Date: Feb. 12, 2015

(54) DEVICES FOR PORTABLE AIRWAY PRESSURE SYSTEMS

(71) Applicants: Thomas G. GOFF, Mountain View, CA (US); Nathaniel L. BOWDITCH, Menlo Park, CA (US); Kirby CHIANG, Los Altos, CA (US)

(72) Inventors: Thomas G. GOFF, Mountain View, CA (US); Nathaniel L. BOWDITCH, Menlo Park, CA (US); Kirby CHIANG, Los Altos, CA (US)

(21) Appl. No.: 14/455,139

(22) Filed: Aug. 8, 2014

Related U.S. Application Data
(60) Provisional application No. 61/864,191, filed on Aug. 9, 2013.

(51) Int. Cl. A61M 16/08; A61M 16/20

(52) U.S. Cl. CPC A61M 16/0875; A61M 16/208; A61M 2205/0216
USPC 128/205.24; 128/204.18

(57) ABSTRACT

Devices and components related to positive air pressure (PAP) systems, including portable PAP systems, are provided. A portable hose for a PAP system can comprise a flexible conduit at least partially surrounded by a fluid expandable support member.

### CROSS REFERENCE TO RELATED APPLICATIONS

This application claims the benefit of U.S. Provisional Patent Application No. 61/864,191, filed Aug. 9, 2013, entitled “DEVICES FOR PORTABLE AIRWAY PRESSURE SYSTEMS”, the entire disclosure of which is hereby incorporated by reference.

### BACKGROUND

Obstructive sleep apnea (OSA) occurs when tissue in the upper airway blocks the airway during sleep. The brain will sense the rise in CO2, and will wake up the person so that breathing resumes. Such an event is called an apnea. A partial airway blockage causing an awakening is called a hypopnea. A person is unlikely to remember such awakenings, but sleep is disrupted. The severity of obstructive sleep apnea is measured by the frequency of awakenings, as shown in the table below.

| Apneas + Hypopneas/Hour | OSA Classification |
|---|---|
| 0-5 | Normal |
| 5-15 | Mild |
| 15-30 | Moderate |
| 30+ | Severe |

Untreated, OSA not only leaves patients chronically fatigued, but it also carries significant cardiovascular consequences.

Positive Airway Pressure, or PAP, is the most widely used and the most effective treatment for OSA. In PAP, a bedside compressor supplies pressurized air to the patient's airway through a hose and mask. The air pressure is set sufficiently high to maintain an open airway during sleep. Examples of PAP devices may be found, e.g., in U.S. Pat. No. 8,316,848; U.S. Pat. No. 8,453,640; and U.S. Pat. No. 8,353,290, the disclosures of which are incorporated herein by reference.

Many OSA patients who use PAP have difficulty using their PAP systems when traveling. Most PAP systems are both bulky and too fragile to pack in checked luggage. For travel, patients prefer small, light PAP systems. Despite recent introduction of some portable PAP systems, there remain significant shortcomings in their design.

### Lack of Anchoring

Many patients will keep a second, smaller PAP system just for travel. But there is a downside to decreasing size. A smaller, lighter PAP system is more likely to get pulled off a bedside table when tugged by the hose connecting the console to the patient's mask. Some small PAP systems have some sort of heavy docking station to prevent the PAP system from being pulled off the bedside table. However, such docking systems add weight and bulk, which may cause patients to forego them when traveling.

### Bulk of the Airway Hose

The airway hose commonly used in PAP systems is quite large to pack and transport. In many cases, the hose itself is larger in volume than the PAP flow generator unit. This significant volume inhibits travel and portability of the entire system. As the airway hose is a necessary component of the typical PAP system, it is required for most use. Standard hose technology has not evolved significantly in many years. Typical construction consists of an inner membrane of plastic, surrounded by a spiral of thicker, stiffer plastic to give the hose structure. This structural surrounding is intended to prevent the kinking and crushing of the hose in use. This structural element also inhibits portability.

### Cumbersome Humidification

Smaller, more travel-friendly CPAP machines are being introduced to the market. However, they either lack humidification or, if they include it, it requires extra bulk. Many travelers leave their humidification systems at home when they travel. The humidification units for many PAP systems are just as large as the flow generator. Humidification units are comprised of a large reservoir for holding water, and technology to convert the fluid water into a mist or vapor. The bulk of the water chamber is not compressible, and therefore inhibits portability and travel.

### SUMMARY OF THE DISCLOSURE

In some aspects a hose for a portable PAP system is provided. The hose comprises a flexible air conduit; and a fluid expandable support member positioned around and extending longitudinally along at least a portion of the flexible air conduit, the fluid expandable support member having an expanded state and a collapsed state.

The fluid expandable support member can comprise at least two radial wraps connected by a longitudinal portion. The fluid expandable support member can comprise a tubular shape helically wound around the flexible air conduit. In some embodiments, the flexible air conduit comprises plastics, plastic materials, elastomers, thermosets, polymers, fabrics, woven fibers, woven plastics, heat-treated materials, and material composites. The flexible air conduit can comprise a diameter of about 12-25 mm when the fluid expandable support member is in the expanded state. In some embodiments, the hose weighs about 10-100 g when the fluid expandable support member is in the collapsed state. The hose can have a volume of about 50 cc when the fluid expandable support member is in the collapsed state. The hose can further comprise a valve. The valve can comprise an actuator configured to allow airflow from the flexible air conduit to the fluid expandable support member when pressed.

In other aspects, a hose for a portable PAP system is provided. The hose comprises a flexible air conduit; and a fluid expandable support member helically wound around the flexible air conduit, the fluid expandable support member having an expanded state and a collapsed state.

The support member can comprise narrow portions and wide portions.

In other aspects, a method of using a hose for a portable PAP system is provided. The method comprises inflating a fluid expandable support member positioned around and extending longitudinally along at least a portion of a flexible air conduit, thereby changing the flexible air conduit from a collapsed state to an at least partially open state.

Inflating can comprise providing fluid to the support member via a valve positioned on the support member. In some embodiments, inflating comprises pressing an actuator on a valve positioned on the support member. Inflating can comprise opening a valve positioned on the support member, thereby allowing air to flow from the air conduit to the support member. The method can comprise sealing an end of the air conduit during inflation of the support member. In some embodiments, the method comprises connecting the hose to a pump of a portable PAP system. Inflating can comprise providing air to the support member.

### BRIEF DESCRIPTION OF THE DRAWINGS

FIG. 1 illustrates an embodiment of PAP system comprising suction elements.

FIG. 2 illustrates an embodiment of a PAP device comprising a hook and loop attacher.

FIG. 3 illustrates an embodiment of a PAP device attached to a bed frame.

FIGS. 4A-6 illustrate embodiments of PAP devices comprising anchoring tongue attachers.

FIG. 7 illustrates an embodiment of a PAP device secured into a wall outlet.

FIG. 8 illustrates an embodiment of a PAP device attached to the underside of a bed frame.

FIGS. 9A-E illustrate embodiments of a PAP device enclosed in an inflatable sleeve.

FIGS. 10-11 illustrate embodiments of a PAP system incorporating a portable humidifier.

FIGS. 12A-C illustrate an embodiment of a portable hose.

FIG. 13 illustrates an embodiment of a portable hose.

FIG. 14 illustrates an embodiment of a portable hose.

FIG. 15 illustrates an embodiment of a valve.

### DETAILED DESCRIPTION

The current invention uses various means to secure a PAP console in the sleeping environment. This will help prevent the console from being moved by tugging of the hose when the patient is wearing the system. Several attachers or means for securing a PAP console are disclosed. Most are reversible. In some embodiments, an attacher is positioned on the PAP device. The attacher can be used to fix the PAP device to a surrounding surface. In some embodiments, a first attacher is positioned on the PAP device, and a second attacher is attached to a surrounding surface. The first and second attachers can be configured to be reversibly attached to one another. Different mechanisms and means may be more appropriate for different sleeping environments, depending on the fixturing surfaces available.

Surfaces to which a PAP device can be secured in typical sleeping environments include: walls, ceiling, floor, headboards, bed frames, bed side boards, underside of beds, bedside tables, electrical outlets, lamps, bedposts, mattresses, pillows.

### Suction

Suction is an embodiment of an attacher that can be used to reversibly adhere a PAP device to a bed or bedside surface. Such suction can be achieved manually using a suction device on the PAP console that the patient engages. Suction can be achieved using one suction structure, or a multitude of suction structures. Suction can be created by pressing the PAP device onto a surface, forcing the air out of the suction elements. Alternatively, a manage device, for instance a pump or syringe, can be used to remove air from the suction elements. In another embodiment, suction is provided by an array of suction structures located on the underside of the PAP device.

Alternatively, suction can be created and/or maintained using the compressor within the PAP console. In this latter system, there can be communication, either temporary or permanent, between a PAP air inlet passage and an area on the surface of the PAP console in which a lower pressure is created to achieve suction adhesion.

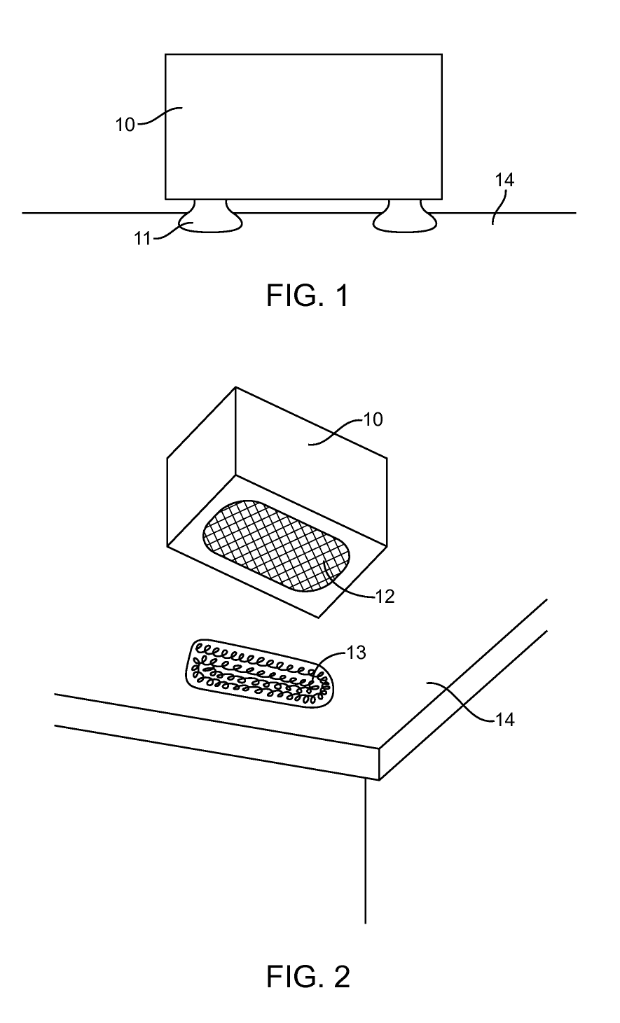

FIG. 1 is a side view of a PAP device 10 with suction elements 11 extending from the PAP device 10 and attaching it to a surface 14 in the sleeping environment. The one or more suction elements 11 can provide a typical suction force. Alternatively, the suction elements 11 can provide suction through the application of an active vacuum (not shown).

### Clip and Hook Elements

In another embodiment, attachers can comprise clip and/or hook elements that are provided to secure the PAP device to the bed, headboard, sideboard, or bedside table. These elements can be made of bent wire, metal, plastics, or similar materials. These elements are used to hang the PAP device securely but reversibly in the sleep environment. Hooks are used to hang the PAP device using gravity to provide the force to keep the device in place.

### Hook and Loop

In another embodiment, hook and loop fasteners are used as embodiments of attachers to attach the PAP device securely to a surface in the sleeping environment. One side has the hook structure, consisting of many small hooked elements, densely arranged. The other side has the loop structure, consisting of many small looped elements, densely arranged. One side of the hook and loop fastener is placed on the PAP device. Adhesives or a physical capturing mechanism can be used to achieve this. The other side of the hook and loop fastener is placed on the bed or bedside surface to which one would like to attach the PAP device. The PAP device can be repositioned at the hook and loop interface. In a preferred embodiment, the hook side of the combination is attached to the PAP device such that the device can be attached to various bedding and bedside materials, such as blankets, carpeting, table cloth, or similar fabrics, which inherently function as the loop fastener without necessitating the addition of a separate loop surface. In one embodiment, hook fasteners are attached in multiple areas on the underside of the PAP device using adhesive. Loop fasteners are attached to a surface in the sleeping environment using adhesives as well. In one example, these loop fasteners could be minimal in size and could be attached to the top surface of a bedside table, to prevent the movement of the device when the airway tube is pulled.

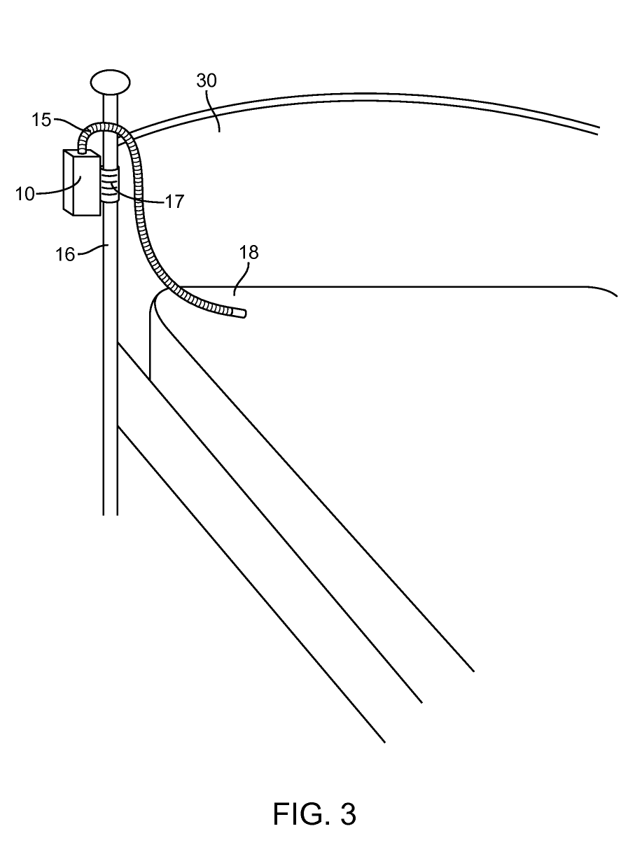

FIG. 2 is a perspective view showing the underside of a PAP device 10 with a surface of hook fastener 12 applied to it. FIG. 2 also shows a surface 14 in the sleeping environment with a corresponding surface of a loop fastener 13 applied to it. When the hook surface 12 is brought into contact with the loop surface 13 the PAP device is secured to the surface 14 in the sleeping environment. The hook and loop surfaces may be applied to either the PAP device or the sleeping environment surface. They may be placed in a vertical or horizontal arrangement.

### Clamps

In another embodiment, a clamp attacher provides the means to attach the PAP device in the sleeping environment. The clamp can attach to a bedpost, headboard, sideboard, bed frame, lamp, or other sleeping environment object. The clamp can be easily attached and detached from the PAP device via a quick release design. Clamps can be actuated using CAM devices, spring loaded, or threaded posts.

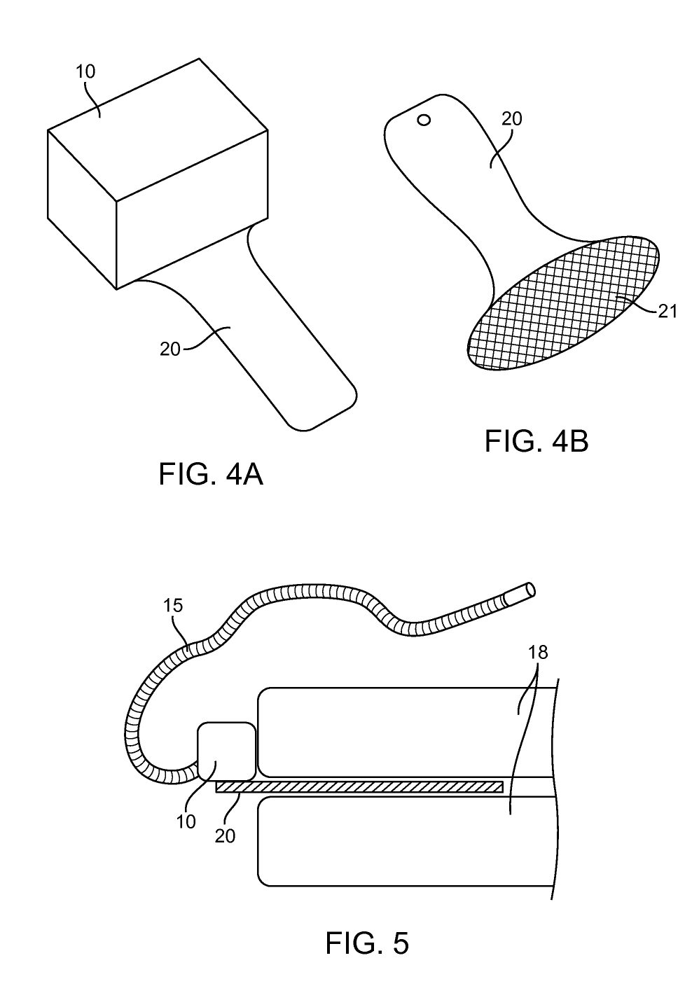

FIG. 3 shows a PAP console 10 attached to a bed frame 30. Specifically, the PAP device 10 is attached to the bed post 16 of the frame 30 using a clamp 17 extending from PAP console 10. The PAP device 10 is secured from movement, and can easily be removed for transport. Alternatively, the clamp can extend around both the PAP console 10 and the bed post 16 (not shown).

### Anchoring Tongue

In another embodiment, an anchoring tongue attacher is attached to the PAP device. This tongue apparatus is relatively thin. In some embodiments, it has a thickness between 0.020″ and 0.500″. It can be long and narrow or short and wide, or some combination thereof. Increasing the surface area of the tongue apparatus increases its holding ability. The surface area provides the friction which resists movement forces placed on the PAP device. The tongue apparatus is relatively rigid. It is placed horizontally between two mattresses, between a mattress and a box spring, between a mattress and bed slats, or between a mattress and a bed frame to secure the PAP device. It can also be placed vertically between a mattress and a sideboard or headboard of a bed. The tongue-like apparatus can be easily attached and detached from the PAP device via a quick release mechanism, described elsewhere herein. This design is lightweight and secure. As it is very thin, it can easily be packed for travel. In some embodiments, the tongue-like anchor has a tacky surface treatment that helps to prevent it from sliding. In further embodiments, the tongue anchor device can be made to be collapsible, such that it can fold up into a smaller form for transport, and then unfold for use. For instance, in one embodiment the anchoring tongue could form the shape of a “T”, with a joint at the intersection of the two parts. The upper part is able to rotate into a locked position orthogonal and be placed between two mattresses. For travel, the upper part is rotated to be parallel with the lower part. In some embodiments, the anchoring tongue is sized to fit conveniently in a laptop case or at the bottom of a suitcase so that it is easy to transport for travel.

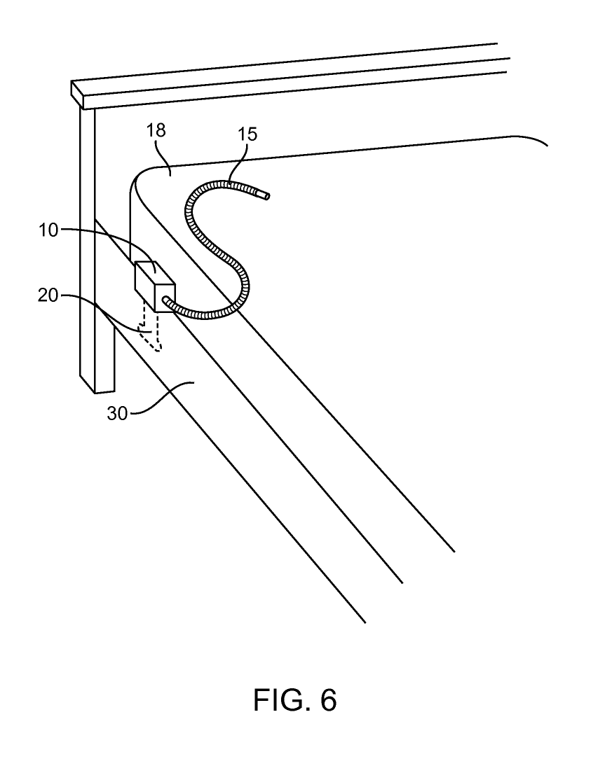

FIGS. 4A and 4B show an embodiment of the anchoring tongue 20. FIG. 4A shows the anchoring tongue 20 element elongated and attached to a PAP device 10. The tongue 20 can be placed some distance into the space between two mattresses. FIG. 4B shows an embodiment of the anchoring tongue 20 without the PAP device attached, and with an optional shaded area of enhanced surface friction 21. This added friction area 21 could be in a specific portion of the anchoring tongue 20, or cover most of its surface. The increased friction area can be achieved with adhesive or surface treatments which enhance the amount of friction between the anchoring tongue and surfaces and materials common to the sleeping environment.

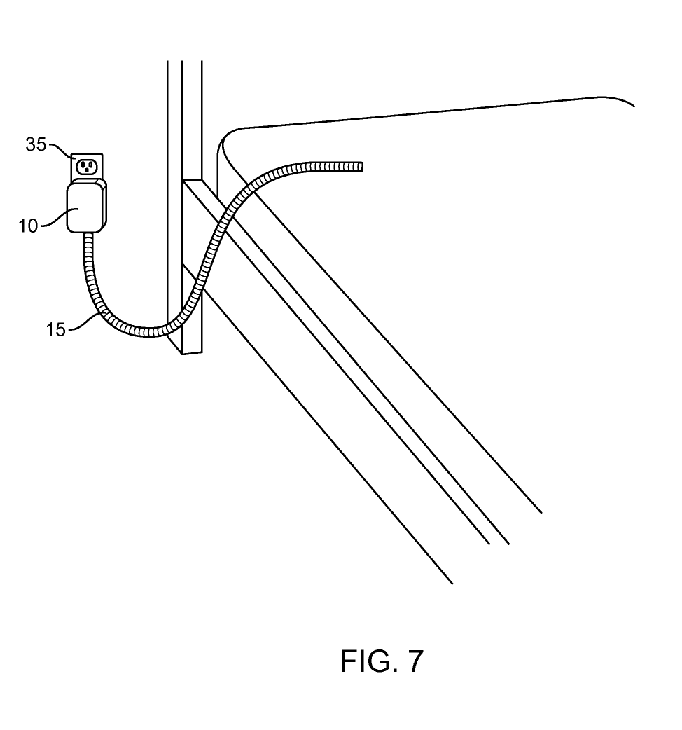

FIG. 5 illustrates the anchoring tongue 20 in use, placed between two mattresses 18. In the figure, the anchoring tongue 20 extends a length of about 3-5 times the width of the PAP device 10 into the space between the mattresses 18. This length is only representative, and may be varied considerably. The PAP device 10 is securely attached to the anchoring tongue 20. The PAP device 10 and tongue assembly is held securely by the friction forces over the surface area of the tongue 20 such that normal forces exerted on the PAP device 10 during typical use do not dislodge it from the bed.

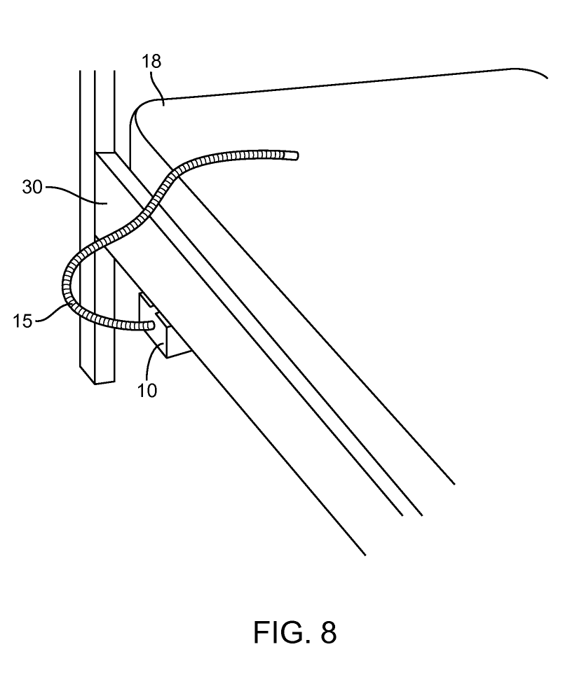

FIG. 6 shows a PAP device 10 attached to the anchoring tongue 20, with the anchoring tongue 20 placed vertically between the mattress 18 and the bed frame 30 in the sleeping environment. In this embodiment, PAP device 10 is attached to anchoring tongue 20. This is another arrangement to secure the PAP device in place.

### Direct Plug-In to Outlet

In another embodiment, the PAP device is configured to plug directly into a wall outlet. This provides the support and location of the PAP device. The prongs of the electrical leads on the PAP device act as an attacher and are placed directly into the wall outlet, holding the device securely in place. The lightweight nature of travel devices makes this approach even more secure. Although this approach requires a wall electrical outlet in proximity of the head of the bed, this is the case in most sleeping environments, where the outlet is already powering alarm clocks, bedside lamps, etc. This feature has the advantage of simplifying the setup and fixturing of the PAP device. Additionally, no power cord would be required, which is a significant advantage when traveling.

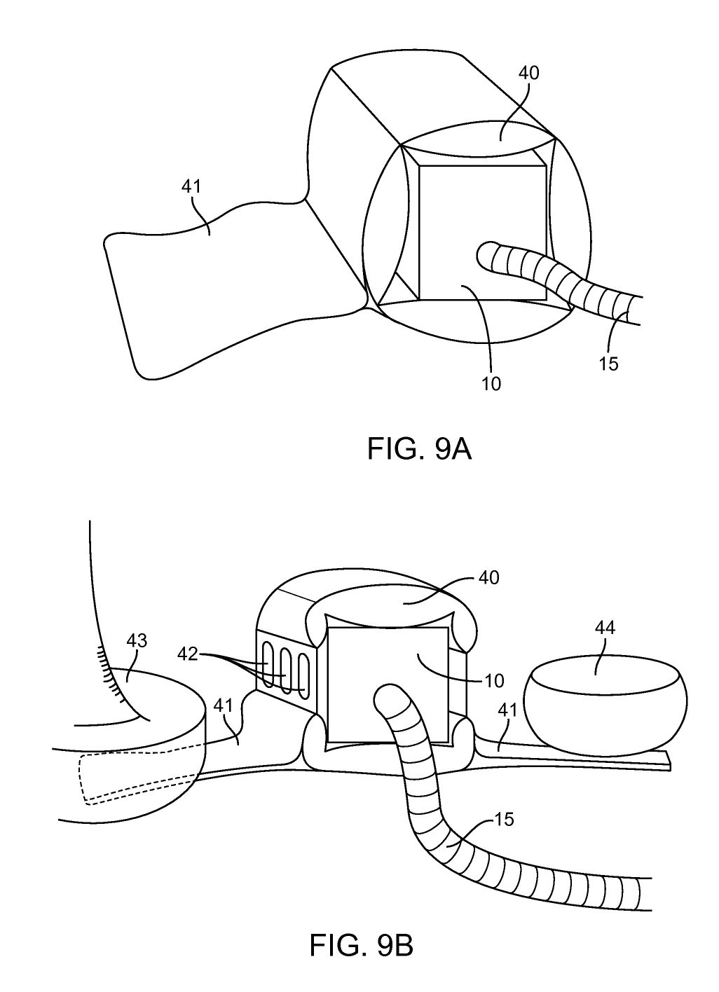

FIG. 7 illustrates a PAP device 10 which is secured directly into a wall outlet 35 for use. There is no power cord needed. The prongs extend from PAP device into the wall outlet 35, and the connection between the prongs and the outlet is sufficient to support the lightweight PAP device 10 and hold it securely against any forces exerted on it during sleeping. Such a system can have a two or three prong plug interface. The tube shown exiting the PAP device is the airflow conduit 15.

### Hang Under Bed

In another embodiment, the PAP device is hung from the underside of the bed. Attachers such as hooks can be used to hang the PAP device from the bed frame or mattress. Hook and loop fasteners, or a mounted plate can be used to secure the PAP device under the bed. This attachment means has the advantage of keeping the PAP device out of sight, but also off the floor. CPAP users sometimes place their machines on the floor under their beds. However, the floor underneath beds is commonly neglected and infrequently cleaned, and thus accumulates dust, hair, and other undesirable material for a respiratory environment. Hanging the PAP device off the ground separates it from this environment.

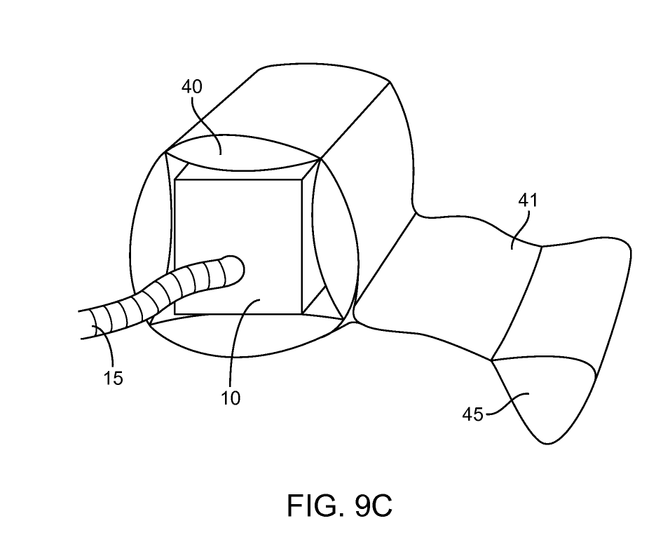

FIG. 8 shows the PAP device 10 suspended under the bed. It is hanging from the underside of the bed frame 30. It may be attached using attachers such as clips, screws, snaps, hooks, hook and loop fasteners, adhesive, straps or similar attachment means. Note the distance between the PAP device and the floor. The tube shown exiting the PAP device is the airflow conduit 15.

### Inflatable Element

In another embodiment, an inflatable member provides the support to cradle the device, holding it securely in place while also insulating sound and vibration when inflated. This inflatable member is secured to the sleeping environment using any of the methods described herein. When not in use, such as for travel, the inflatable member is deflated and takes up very little room. It can have its own inflation/deflation pump integrated into the member, or a separate pump to use to inflate and deflate the member. This configuration offers several advantages. All PAP devices create noise and vibration. The inflatable element helps limit those. Further, the inflatable element snugly holds the PAP device. In one embodiment, the inflatable element is tubular in form, allowing the device to be placed within it. In another embodiment, the inflatable element forms a cavity when inflated, and the device fits into the cavity.

The inflatable element can be secured by means described elsewhere herein to the sleeping environment. Or, the inflatable element can secure both the device, and inflate around an element in the sleeping environment to hold it in place. This could be done around a bedpost or lamp, for example.

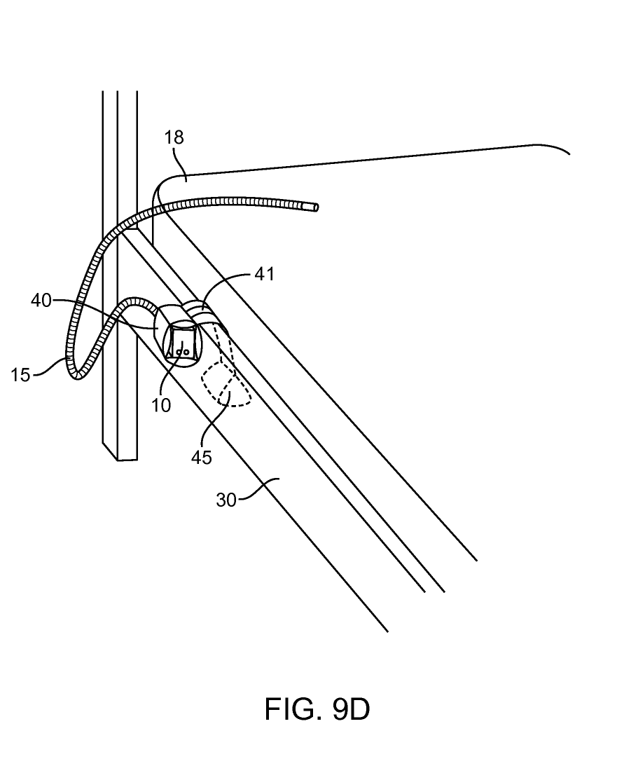

FIGS. 9A, B, C, D, and E show a PAP device 10 enclosed in an inflatable sleeve 40, with various attachment means. FIG. 9A shows a PAP device 10 enclosed in an inflatable sleeve 40. The inflatable sleeve 40 secures the device once it is inflated, holding it in place and helping to isolate sound and vibration. In this embodiment, there is an extension 41 of the inflatable sleeve 40, which can be used as an attacher to secure the device and sleeve to a surface in the sleeping environment. The extension 41 can help secure the device 10 and sleeve 40 by the placement of weighted objects on top of it. In another embodiment, the extension can be lined with a tacky, reversible adhesive to attach it to a surface 14 in the sleeping environment. The sleeve can be deflated for transport.

FIG. 9B shows a PAP device 10 enclosed in an inflatable sleeve 40, with two sleeve extensions 41 extending from the sleeve 40. These extensions 41 are shown held in place by weighted objects, shown here as a bedside lamp 43 and a small heavy object such as a stone or bowl 44. In some embodiments, the inflatable sleeve 40 may be fenestrated, such that the fenestrations 42 allow for the passage of air to the PAP device 10.

FIG. 9C illustrates the inflatable sleeve 40, with an extension 41 leading to an inflatable or finable anchor 45. This anchor 45 can be placed into a small space and then inflated such that the inflation pressure holds the anchor 45 in place. In another embodiment, the anchor 45 can be filled with a fluid, e.g. water, which provides the weight to keep it in place.

FIG. 9D shows the PAP device 10 secured in the inflatable sleeve 40, with the extension 41 draped over the side rail 30 of a bed, and the anchor 45 inflated and wedged in between the side rail 30 of the bed and the mattress 18, holding the system securely in place.

FIG. 9E shows the PAP device 10 secured in the inflatable sleeve 40, with the extension 41 draped over the edge of a drawer 46 of a bedside table. The anchor 45 is held inside the drawer 46 once it is closed, thereby keeping the system in place throughout the night.

### Elastic Band

In another embodiment, the PAP device is secured to a surface in the sleeping environment using an elastic band as an attacher. The elastic band can be attached to a bedpost. The elasticity of the band allows it to fit securely around materials of different dimensions, making it very versatile for the traveler. The elastic band can be provided with a pocket for the device. Once the device slips into this pocket, it is securely held in place by the elasticity of the material. The band can have two function loops: one to attach to a sleeping environment element, and one to allow for the insertion and removal of the device. The loop for the insertion of the device can be made of an air-permeable fabric, such as a mesh with open loop weaving. This material will allow the flow of air to the device during use. Alternatively, the elastic band can attach to the device in 2 or more places. These attachment points could be enhanced with quick connect clips. The ends of the elastic band could have fastening elements shaped to fit into the receiving elements on the device, securing the elastic band to the device.

### Adhesive

In another embodiment, adhesives can be used as an attacher to secure the PAP device in the sleeping environment. In one embodiment, the adhesive used can be of sufficient bond strength to prevent sliding and movement of the PAP device during normal use, but insufficient to prevent the user from removing and repositioning the device as desired. This adhesive could be permanently attached to the PAP device, and adhere to typical bedside surfaces like a bedside table. In one embodiment, the adhesive has a lower tack force, designed to resist lateral forces more than perpendicular forces. Alternatively, a gel pad could be placed between the PAP device and the bedside table to prevent lateral movement. This gel pad or pads could be tacky to a matched gel surface on the bottom of the PAP device, but not tacky to other materials common to the sleeping environment.

### Magnets

In another embodiment, magnets are used as attachers to secure the PAP device in the sleeping environment. One magnet is mounted on the PAP device, preferably on the underside. The other magnet is attached to a surface in the sleeping environment. The PAP device can be reversibly attached to the sleeping surface with the magnetic force. With a powerful magnet, such as a neodymium magnet, placed under the top surface of the bedside table, and held in place with adhesive, tape, or the like, the device could be held securely in place on the bedside without any visible trace of the securing mechanism when the device is removed. This offers significant aesthetic advantages, particularly for users who do not wish to display their PAP devices in their bedrooms when not in use.

### Flexible Ratcheting Mechanism

In another embodiment, a flexible ratcheting element is used as an attacher to attach the PAP device to the sleeping environment. The element includes a band with steps or teeth formed in it. The band is inserted into a ratcheting fitting, which allows it to slip into place, and tighten as each step is passed through the ratchet. When desired, a separate element is activated to reduce the contact between the ratchet and the teeth, so that the mechanism is loosened and/or removed.

The mechanism can be reusable or disposable. In a preferred embodiment, the mechanism is reusable. In one embodiment, the mechanism is disposable and must be cut to be removed. In one embodiment, the flexible ratcheting mechanism is made of an elastomeric material, and includes a line of holes through which a post, also from the band, can be placed to secure the device in place.

### Hook Into Mattress

In another embodiment, a hook or a series of hooks acts as an attacher, and is secured to the PAP device by a removable means. The hook(s) penetrate the outer layer of the mattress or other soft material without damaging the material in a way that would negatively affect its use. In one embodiment, the hooks are thin enough to pass without disturbing the weave of the fabric, but not so sharp as to penetrate human skin under normal use. The hooks can also be hooked in between the mattress and another bedroom furnishing, such as a box spring or bed frame, for added holding strength. The hooks can be made from thin plastic with blunt tips for safety. Such a design can pass through the fabric layer covering mattresses and bedding, but would not penetrate human skin under similar forces.

### Attaching to the PAP Device

Several approaches are provided for attaching the PAP device to the fixturing means. One or more threaded posts could be used to quickly secure the PAP device to any of the herein described fixturing means. Further, a combination of threaded posts and non-threaded posts may be used, such as a single non-threaded post for resisting rotation and a single threaded post for secure attachment.

### 90-Degree Spin Slot and Post

Another manner of quickly and reversibly attaching the PAP device to the fixturing means is by way of posts and slots. A 90 degree turn of the post within the slot can push it beyond a detent and secure it into place.

### Cradle

In another embodiment, a cradle is provided to secure the PAP device in place. The cradle can then be attached to various attachment means. The cradle contains a degree of flexibility which allows it to grasp the PAP device and hold it securely with the tension of the cradle.

### Humidification

This device comprises a small apparatus that is specially adapted to interface with the standard threading on widely available drinking water bottles. In this way, the user is not required to travel with a water reservoir. Water bottles can be found nearly anywhere one might travel. With the standard fitting, (such as SPI 28MM thread specs), simply procuring one of these bottles at the destination thereby outfits the user with a full humidification system. The fitting can be designed to fit the majority of flatwater bottles in the marketplace. Additional fittings and adaptors can be provided to fit different thread designs for different bottles in different markets. Further, once done with the CPAP and humidifier, the water bottle that has served as the reservoir can simply be discarded or recycled. This significantly reduces the amount of material volume and weight with which the CPAP user must travel. In one embodiment, the micro-humidifier is integrated into the CPAP base unit.

In another embodiment, the micro humidifier is an attachment unit that can be connected to any CPAP machine to provide humidity to the airflow. This connection could occur through the classic tubing fittings. The humidification unit can work using several different mechanisms well known in the art, including: evaporation, steam, ultrasonic, diffuser. The unit can be powered through a standard wall plug or with batteries.

In a further embodiment, the humidification unit to which the bottle is attached can deliver its humidified air through a small tube that is connected to the tubing or mask interface near the patient to humidify the air.

The bottle could be connected to sit upright, upside down, or lay on its side. It could have a tube extending into it for the sourcing of the water. Bottles of various sizes could be used with the same standardized fitting. The bottle and device could also be fashioned to conveniently attach to the sleeping environment. They could attach to the headboard, sideboards, mattress, under the bed, side table, lamp or other attachments means.

In a further embodiment, the humidification apparatus can also heat the water, providing heated humidification.

In a further embodiment, a kit is provided with multiple methods for securing a PAP device. The most appropriate method can be chosen depending on the sleeping environment conditions, which may vary nightly during travel.

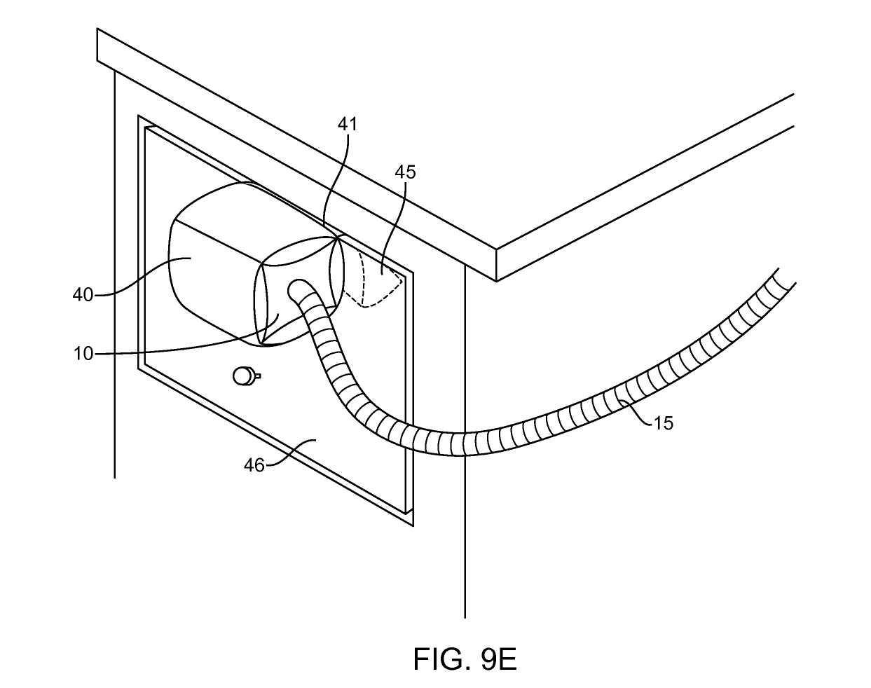

FIG. 10 shows a PAP system 10 incorporating the portable humidifier 50, threaded connection 61, and water bottle 60 described above. The water bottle 60, which serves as the water reservoir, could be used standing up as shown. The water bottle could also be used lying on its side. A tube extending into the bottle can be weighted to reach the lowest point of the fluid.

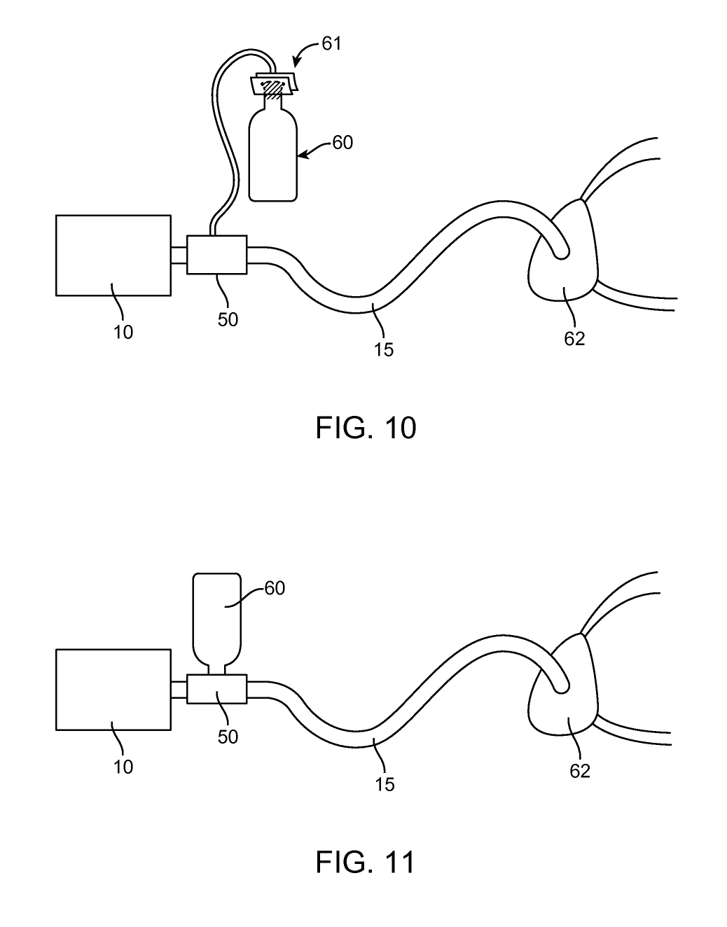

FIG. 11 shows a PAP system 10 incorporating the portable humidifier 50 with integrated connection, and water bottle 60 described above. Here the water bottle 60 is depicted inverted. This allows the water to drain from the bottle via gravity into the humidification element 50. The humidification element 50 has a threaded female opening (not shown) mating with the threaded opening of the water bottle.

Collapsible Airway Hose

Described here is a collapsible hose that takes up minimal space when not in use. In some embodiments, the hose comprises a flexible conduit (e.g., a thin tube), which is the sealed conduit for airflow. The hose can comprise a generally circular or ovular cross section. Other configurations are also possible (e.g., rectangular). Though it forms the barrier and corridor for the airflow, the tube lacks structure. Around this conduit is a second element, which is a support member that provides the structure for the hose. In some embodiments, this structured portion includes an expandable or inflatable chamber. This support member can be tubular and comprise a generally circular cross section. In other embodiments, the cross section is not circular. For example the cross section can be ovular or rectangular. The support member can be wound in a spiral fashion around the outside of the sealed conduit. The support member has a port for the introduction of fluid (e.g., air) into its chamber, perhaps at one end of the tube. This port could be augmented with a one-way inlet valve to prevent back flow. This valve allows the inflow of air from the flow generator or other source. The air fills the support member, bringing it into a fully expanded (or partially expanded) state from a collapsed (or partially collapsed). Alternatively, instead of a one-way valve, this part could be a stop cock or similar controlled valve, which is actuated by the user. Once the inflatable chamber is filled, the control valve can be actuated to shut off the inflatable chamber. Optionally, at the other end of the inflatable chamber another valve can be placed to allow for the exit of the fill air at the discretion of the user.

In some embodiments, the user wraps the deflated tube up into a small ball of deflated material, which could be smaller than a fist. The deflated tube can be transported easily and takes up very little room. The deflated tube can also be lightweight. When desired, the tube is connected to the flow generator. The airflow fills up the structure bladder of the tube. This can be aided by blocking the distal opening of the tube with the hand or other means. Once the structure bladder is inflated, the entry valve can be closed off Or, if it is the one-way valve, it will close itself off Now the tube has structure and is ready to function as a normal CPAP tubing conduit for airflow. Because of the closed off air structure chamber, it is resistant to kinking and blockage. Flexibility of the hose can be altered by material choice, spacing and geometric arrangement of the air chamber, and inflation pressure. Further, a fluid such as water could be used to inflate the structural element to provide greater resistance to kinking. Additionally, a combination of inflation fluids could be used, with lighter weight air near the user and more durable liquid near the flow generator.

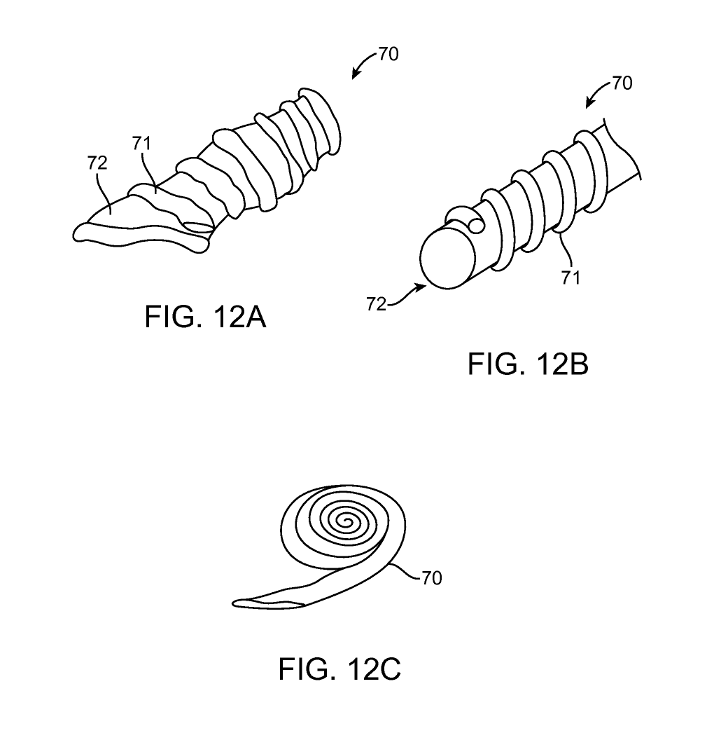

FIGS. 12A, B, and C illustrate one embodiment of the portable hose concept. An inflatable structure chamber 71 around the portable hose 70 is shown. FIG. 12A shows the hose deflated, where the structure chamber 71 is deflated as well as the air conduit 72. FIG. 12B shows the same view of the tube when inflated and in service. The structural chamber 71, when inflated, expands the air conduit 72 and assumes a helical geometry around the hose to support it. It remains flexible for bending. FIG. 12C shows the deflated hose 70 wrapped into a tight coil for portable transport.

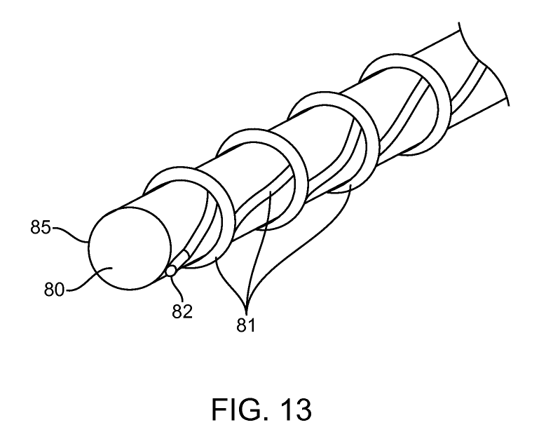

FIG. 13 illustrates an embodiment of the hose construction. The air flow lumen 80 is surrounded by a thin air barrier 85. An inflatable structural element 81 on the outside of the air barrier 85 provides the structure for the airway tube. Once inflated, it can resist kinking and collapse of the airway tube, but allow for bending. The structural element 81 is constructed to radially wrap around the air flow lumen 80, while also having longitudinal communication through longitudinal portions between its radial wraps. A valve 82 is provided to allow for the inflation and deflation of the structural element 81. In some embodiments, more than one valve can be provided (e.g., 2, 3, 4). When deflated, the entire assembly collapses to occupy a very small amount of space.

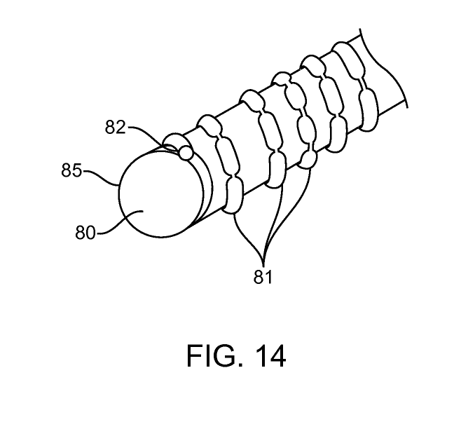

FIG. 14 illustrates another embodiment of the hose construction. The air flow lumen 80 is surrounded by a thin air barrier 85. An expandable structural element 81 on the outside of the air barrier 85 provides the structure for the airway tube. The structural element 81, can be inflated with fluid (e.g., air) to assume its designated geometry. Once inflated, it resists kinking and collapse of the airway tube, but allows for bending. The structural element 81 is constructed to helically wrap around the air flow lumen 80. The structural element 81 is segmented into narrower and wider portions, allowing for flexibility and strength. There is a continuous fluid lumen connecting the narrower and wider portions such that they are in fluid communication. The wider portions provide more structure and strength while the narrow sections allow for greater flexibility in the assembly by enabling bending. Other configurations not shown in FIG. 13 or 14 are also possible. For example, in some embodiments, the structural element 81 can include longitudinal channels running along the length of the air flow lumen 80, the longitudinal channels connected by rings to ensure fluid communication between the channels. The structural element 81 can be positioned around a circumference of the lumen (e.g., in a helical configuration, in a ring configuration) and extend longitudinally along the lumen. A valve 82 is provided to allow for the inflation and deflation of the structural element 81. In some embodiments, more than one valve is provided (e.g., 2, 3, 4). When deflated, the entire assembly collapses to occupy a very small amount of space.

The air flow lumen 81 can have a diameter, when expanded, of about 4-35 mm, with a typical diameter range of 15-22 mm. The thin air barrier 85 can comprise plastics, plastic materials, elastomers, thermosets, polymers, fabrics, woven fibers, woven plastics, heat-treated materials, and material composites. In some embodiments, the thin air barrier 85 has a thickness of about 0.003-0.100″, with typical thickness in the range of about 0.005-0.015″. As noted above, when deflated, the entire assembly collapses to occupy a very small amount of space. For example, in some embodiments, the entire deflated assembly can comprise about 25-150 cc, but typically 50 cc or less. By contrast, the assembly in the inflated state may have a volume of 1000 cc or more. The entire assembly can also be lightweight in the deflated state. In some embodiments, the deflated assembly weighs about 10 g or less, but may weigh up to 150 g or more.

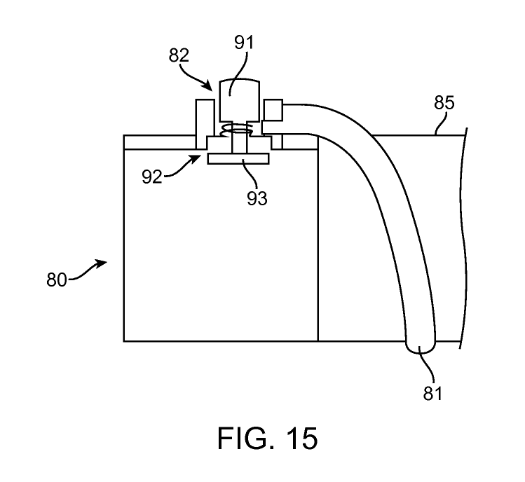

FIG. 15 illustrates an embodiment of the valve 82. The valve 82 includes an actuator 91 which when pressed by the user moves a diaphragm 93 open to allow air flow from the air flow lumen 80 through the valve structure 82 and into the structural element 81. This open valve air path 92 allows the flow generated by the PAP machine to inflate the structural element. The user plugs the distal end of the air flow lumen 80 tubing while the structural element 81 is inflated. Once inflated, the valve actuator 91 is released, moving the diaphragm 93 back into place, thereby sealing off the valve air path 92. When use is complete, the user can deflate the tube for packing. To deflate the structural element 81, the valve actuator 91 is pressed, allowing the air within the structural element 81 to escape. Other configurations of valves are also possible. For example, a stop cock or similarly controlled valve can be used.

Variations and modifications of the devices and methods disclosed herein will be readily apparent to persons skilled in the art. As such, it should be understood that the foregoing detailed description and the accompanying illustrations, are made for purposes of clarity and understanding, and are not intended to limit the scope of the invention, which is defined by the claims appended hereto. Any feature described in any one embodiment described herein can be combined with any other feature of any of the other embodiment whether preferred or not.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A hose for a portable PAP system, comprising:
a flexible air conduit; and
a fluid expandable support member positioned around and extending longitudinally along at least a portion of the flexible air conduit, the fluid expandable support member having an expanded state and a collapsed state.

2. The hose of claim 1, wherein the fluid expandable support member comprises at least two radial wraps connected by a longitudinal portion.

3. The hose of claim 1, wherein the fluid expandable support member comprises a tubular shape helically wound around the flexible air conduit.

4. The hose of claim 1, wherein the flexible air conduit comprises plastics, plastic materials, elastomers, thermosets, polymers, fabrics, woven fibers, woven plastics, heat-treated materials, and material composites.

5. The hose of claim 1, wherein the flexible air conduit comprises a diameter of about 12-25 mm when the fluid expandable support member is in the expanded state.

6. The hose of claim 1, wherein the hose weighs about 10-100 g when the fluid expandable support member is in the collapsed state.

7. The hose of claim 1, wherein the hose has a volume of about 50 cc when the fluid expandable support member is in the collapsed state.

8. The hose of claim 1, further comprising a valve.

9. The hose of claim 8, wherein the valve comprises an actuator configured to allow airflow from the flexible air conduit to the fluid expandable support member when pressed.

10. A hose for a portable PAP system comprising:
a flexible air conduit; and
a fluid expandable support member helically wound around the flexible air conduit, the fluid expandable support member having an expanded state and a collapsed state.

11. The hose of claim 10, wherein the support member comprises narrow portions and wide portions.

12. A method of using a hose for a portable PAP system, comprising:
inflating a fluid expandable support member positioned around and extending longitudinally along at least a portion of a flexible air conduit, thereby changing the flexible air conduit from a collapsed state to an at least partially open state.

13. The method of claim 12, wherein inflating comprises providing fluid to the support member via a valve positioned on the support member.

14. The method of claim 12, wherein inflating comprises pressing an actuator on a valve positioned on the support member.

15. The method of claim 12, wherein inflating comprises opening a valve positioned on the support member, thereby allowing air to flow from the air conduit to the support member.

16. The method of claim 15, comprising sealing an end of the air conduit during inflation of the support member.

17. The method of claim 12, comprising connecting the hose to a pump of a portable PAP system.

18. The method of claim 12, wherein inflating comprises providing air to the support member.
